(19) United States
(12) Patent Application Publication
(10) Pub. No.: US 2004/0230217 A1
(43) Pub. Date: Nov. 18, 2004

(54) Offset trocar piercing tip

(76) Inventor: Peter T. O'Heeron, Houston, TX (US)

Correspondence Address: JACKSON WALKER, L.L.P., SUITE 2100, 112 EAST PECAN ST., SAN ANTONIO, TX, 78205, US

(21) Appl. No.: 10/440,959

(22) Filed: May 15, 2003

(51) Int. Cl. A61B 17/34

(52) U.S. Cl. 606/185

(57) ABSTRACT

A trocar is disclosed which comprises a body assembly and a cannula assembly attached to the body assembly to define a bore therethrough. The disclosed trocar also includes an obturator assembly for sliding engagement in the bore. The obturator assembly comprises a shaft having a central longitudinal axis and having a distal end for insertion into a patient. The distal end of the obturator includes an improved piercing tip which has a substantially flat or planar upper face and lower face which taper away from the shaft to converge at a focal point which coincides with the vertical axis of the central longitudinal axis of the obturator shaft but which is offset from the horizontal axis of the central longitudinal axis of the obturator shaft. The improved piercing tip provides for a clean and controlled insertion of the trocar into the abdominal wall of a patient while minimizing the risk of collateral damage to adjacent tissue and underlying organs.

### BACKGROUND OF THE INVENTION

1. Field of the Invention

The present invention relates to surgical instruments known as trocars which are used in endoscopic surgery to pierce or puncture an anatomical cavity to provide communication with the cavity during a surgical procedure. More particularly, the present invention relates to an improved piercing tip having an offset focus point for the obturator of a trocar.

2. Description of the Prior Art

Endoscopic surgery constitutes a significant method of performing surgeries and has become the surgical procedure of choice, because of its patient care advantages over “open surgery.” One form of endoscopic surgery is laparoscopic surgery, and a significant advantage of laparoscopic surgery over open surgery is the decreased post-operative recovery time. In most instances, a patient is able to leave the hospital within hours after laparoscopic surgery has been performed, whereas multi-day hospitalization is necessary to recover from open surgical procedures. Further, laparoscopic surgery provides decreased incidents of post-operative abdominal adhesions and decreased post-operative pain. Cosmetic results are also enhanced with laparoscopic surgery.

Conventionally, a laparoscopic surgical procedure begins with the insufflation of the abdominal cavity with carbon dioxide. The introduction of this gas into the abdominal cavity lifts the abdominal wall away from the internal viscera. The abdominal wall is then penetrated with a device known as a trocar, which includes a body assembly, a cannula assembly attached to the body assembly to form a bore through the body assembly, and a pointed element called an obturator. The obturator has a piercing tip at its end and slides in the bore of the trocar to pierce the abdominal wall to facilitate penetration of the cannula assembly into the abdominal cavity. After insertion of the trocar through the abdominal wall of the patient, the obturator is removed by the surgeon while leaving the cannula or tube protruding through the body wall. Laparoscopic instruments can then be inserted through the cannula to view internal organs and to perform surgical procedures.

The prime function of a piercing tip of an obturator of a trocar is to facilitate the penetration of the abdominal wall so that the cannula may be inserted into the abdominal cavity. It is significant, however, that this function be accomplished while maintaining a clean and controlled incision and while minimizing the potential risk to adjacent and collateral tissue and underlying organs.

Traditionally, trocar piercing tips have been conical or pyramidal in shape with a “pointed” end (i.e., having a sharp focal point) oriented along the central axis of the obturator. To penetrate the abdominal wall, a surgeon employing a conical or pyramidal trocar tip must apply pressure to the obturator shaft in a direction perpendicular to the abdominal wall. Thus, while such a piercing tip easily passes through the tissue of the abdominal wall, it places adjacent tissue and underlying organs at an increased risk of being incidentally damaged due to the pointed tip and the direction and magnitude of the insertion force applied.

Certain trocars, as disclosed in U.S. Pat. No. 5,817,061 to Goodwin and U.S. Pat. No. 5,591,192 to Privitera, have employed a blunted-blade tip in an attempt to facilitate the penetration and dissection of tissue while limiting the risk of collateral damage to nearby tissue and organs. It has been observed, however, that such tips are prone to failure. In particular, the tip of the trocars disclosed in the '061 and '192 patents can have incidents of snapping off during the insertion of the trocar. It is believed that this failure may be attributable to the blunt shape of the tip and the forces to which the tip is subjected upon insertion.

U.S. Pat. No. 6,478,806, assigned on its face to Taut, Inc., discloses a trocar having an assymetrical bladeless tip. The tip includes an apex (i.e., focal point) which is vertically offset from the central axis of the obturator shaft, an elliptical cross-section, and an exterior surface comprising a curvilinear configuration. (See FIGS. 2 and 6-8; col. 7, line 42 to col. 8, line 26). While such a bladeless tip may reduce the risk of collateral damage to adjacent tissue and internal organs, more penetration force is required to insert the tip through the abdominal wall than with a bladed tip. Moreover, the curved exterior surface and vertically offset apex of the tip require a “rocking” or “twisting” action by the surgeon to achieve penetration making the insertion even more difficult and less controlled.

Accordingly, the surgical medicine industry would find desirable an improved trocar piercing tip which facilitates clean and controlled incisions for penetration of the abdominal wall of a patient while still limiting potential risk to collateral tissue and underlying organs. The present invention, as described and claimed below, advances the art by achieving these goals.

### SUMMARY OF THE INVENTION

The present invention is directed toward an improved piercing tip for an obturator of a trocar for facilitating insertion of the trocar into the abdominal wall of a patient. Particularly, the improved piercing tip provides for a clean and controlled insertion of the trocar into the patient while minimizing the risk of collateral damage to adjacent tissue and underlying organs.

An embodiment of the trocar of the present invention includes a body assembly for handling and manipulation by the surgeon and a cannula assembly which is attached to the body assembly. The cannula defines an axial bore therethrough. A trocar in accordance with the present invention also includes an obturator assembly for sliding engagement in the bore. The obturator assembly comprises a shaft having a central longitudinal axis and having a distal end for insertion into the body of a patient. The distal end of the obturator includes an offset piercing tip having a substantially flat or planar upper face and lower face and an angled segment arranged between the upper and lower faces. The upper face, lower face, and angled segment taper away from the shaft to converge at a focal point which coincides with the horizontal axis of the central longitudinal axis of the obturator shaft but which is offset from the vertical axis of the central longitudinal axis of the obturator shaft.

In another embodiment of the present invention, the focal point of the piercing tip of the obturator is “pointed” (e.g., conical or pyramidal).

In still another embodiment of the present invention, the focal point of the piercing tip of the obturator is “blunt” (e.g., flat, rounded, or bulbous).

In yet another embodiment of the present invention, the angled segment of the tip is “beveled” but has a “flat” or “rounded” (i.e., blunt or non-cutting) peak.

In still another embodiment of the present invention, the offset piercing tip of the obturator is rotatable by the surgeon between: (1) a “right” position where the focus point of the tip is located along the horizontal axis of the central axis and to the right of the vertical axis of the central axis of the obturator shaft, and (2) a “left” position where the focus point of the tip is located along the horizontal axis of the central axis and to the left of the vertical axis of the central axis. This allows the surgeon to make the insertion from the left side or the right side of the patient.

In yet another embodiment of the present invention, the obturator tip is releaseably engaged with the shaft of obturator, so that the same trocar may be used with a variety of piercing tip arrangements.

The foregoing has outlined some of the more pertinent characteristics and objectives of the present invention. These characteristics and objectives should be construed as merely illustrative of some of the more prominent features and applications of the invention.

### BRIEF DESCRIPTION OF THE DRAWINGS

In the accompanying drawings:

FIG. 1A is an exploded top view of an embodiment of a trocar in accordance with the present invention.

FIG. 1B is an exploded side view of an embodiment of a trocar in accordance with the present invention.

FIG. 2A is an enlarged perspective side view of a tip for an obturator in accordance with the present invention.

FIG. 2B is an enlarged axial view of the tip of an obturator in accordance with the present invention.

FIG. 2C is an enlarged left side view of the tip of an obturator in accordance with the present invention. In the event the obturator is rotated 180 degrees by the surgeon, FIG. 2C represents an enlarged right side view of the tip of an obturator in accordance with the present invention.

FIG. 2D is an enlarged top view of the tip of an obturator in accordance with the present invention.

FIG. 2E is an enlarged right side view (opposite the side view depicted in FIG. 2C) of the tip of an obturator in accordance with the present invention. In the event the obturator is rotated 180 degrees by the surgeon, FIG. 2C represents an enlarged left side view of the tip of an obturator in accordance with the present invention.

FIG. 3 is an enlarged view of the tip of an obturator in accordance with the present invention depicting the offset focal point of the piercing tip.

FIG. 4A is a profile view of a surgeon grasping the trocar of the present invention depicting the surgeon preparing to insert the trocar into the abdominal wall of a patient.

FIG. 4B is an enlarged section view of FIG. 4A depicting the offset focal point of the piercing tip as it prepares to penetrate the abdominal wall of a patient.

FIG. 5 is a combined axial and top view of the piercing tip of the present invention being rotated between the right position and the left position.

FIG. 6A is an exploded top view of an embodiment of an obturator having a replaceable piercing tip in accordance with the present invention.

FIG. 6B is an exploded side view of an embodiment of an obturator having a replaceable piercing tip in accordance with the present invention.

### DESCRIPTION OF SPECIFIC EMBODIMENTS OF THE INVENTION

A description of certain embodiments of the present invention is provided to facilitate an understanding of the invention. This description is intended to be illustrative and not limiting of the present invention. These and other objects, features, and advantages of the present invention will become apparent after a review of the entire detailed description, the disclosed embodiments, and the appended claims. As will be appreciated by one of ordinary skill in the art, many other beneficial results and applications can be appreciated by applying modifications to the invention as disclosed. Such modifications are within the scope of the claims appended hereto.

In accordance with the present invention, an obturator tip for a trocar having a focal point which is horizontally offset from the central axis of the obturator shaft is provided.

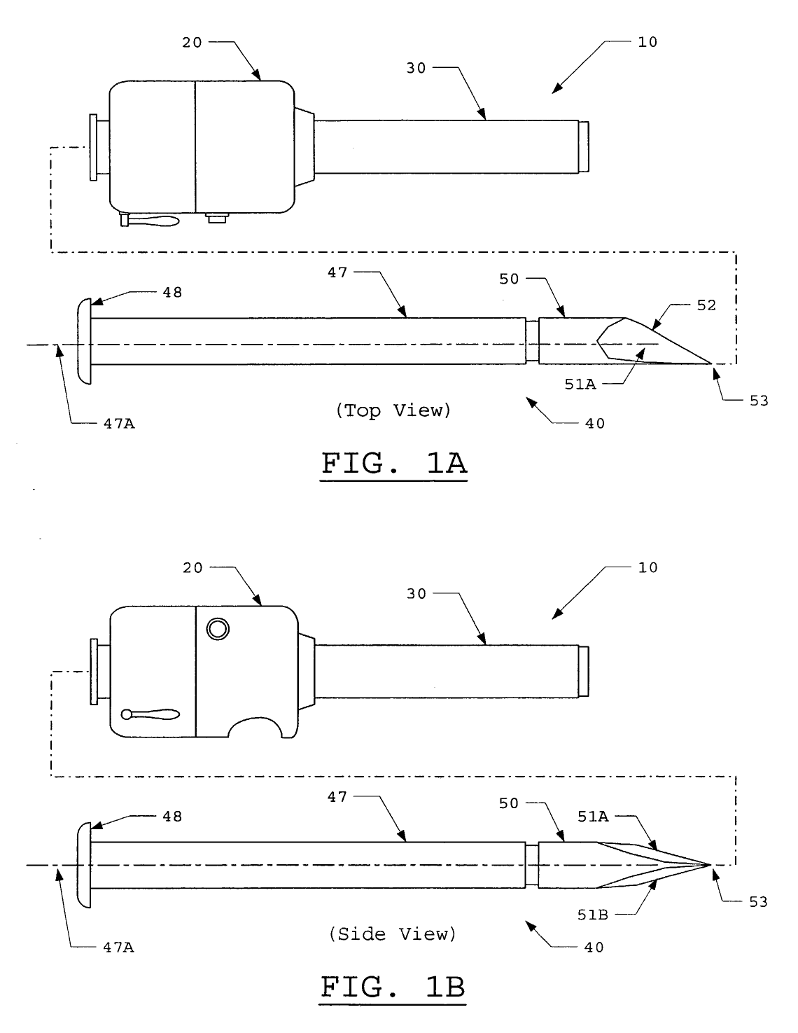

With reference to FIGS. 1A and 1B, a trocar 10 in accordance with the present invention comprises a body assembly 20 to which is attached a cannula assembly 30. The cannula assembly 30 is a hollow tube which defines an axial bore through the trocar 10 when attached to the body assembly 20. A trocar 10 in accordance with the present invention also includes an obturator assembly 40 having a shaft 47 with a central longitudinal axis 47A. The shaft 47 has a proximal end with an arcuate-shaped cap 48 and a distal end with a piercing tip 50 for insertion into a patient. The obturator assembly 40 slides in the axial bore that is defined by the combination of the body assembly 20 and the cannula assembly 30. While the obturator shaft 47 is preferably formed from a stainless steel material, those skilled in the art will appreciate that the obturator shaft may be formed from a variety of materials suitable for use in a surgical environment and capable of withstanding the insertion pressure by the surgeon.

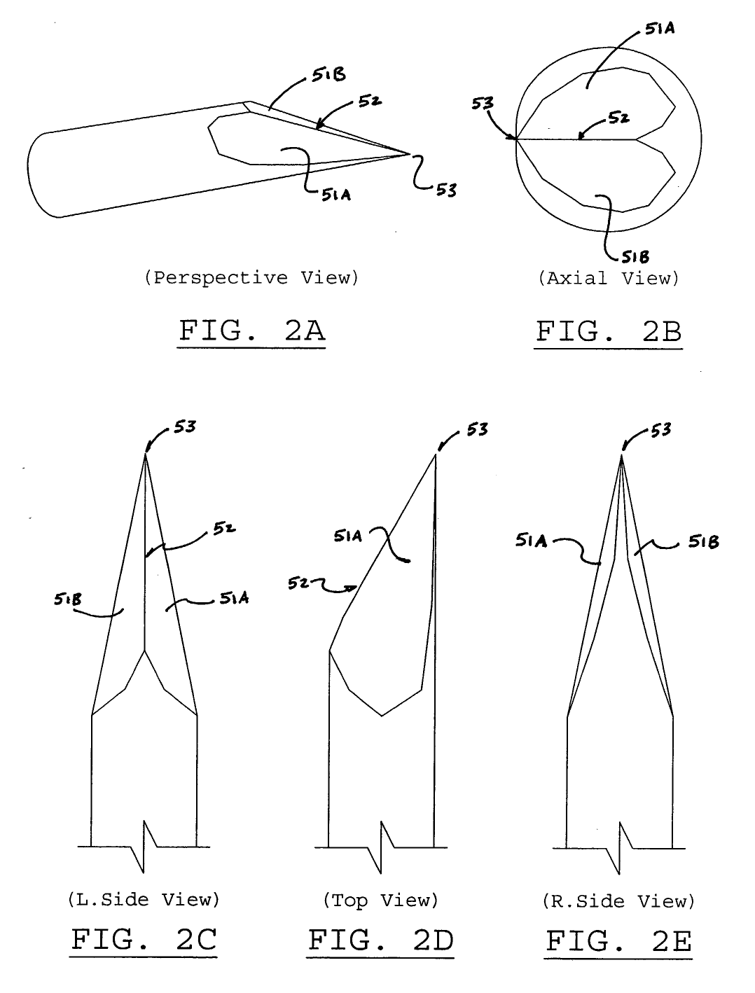

With reference to FIGS. 2A-2E, a preferred embodiment of the piercing tip 50 for a trocar in accordance with the present invention is shown in more detail. The piercing tip 50 comprises a flat or planar upper face 51A, a flat or planar lower face 51B, and an angled segment 52 formed between the upper and lower faces. Preferably, the angled segment 52 is beveled to facilitate tissue separation upon penetration of the trocar into the abdominal cavity, but is not sharpened such that there is no risk of injury to internal tissue and organs. Furthermore, the upper face 51A, lower face 511B, and angled segment 52 of the piercing tip 50 taper away from the shaft 47 of the obturator 40 (see FIG. 1B) and converge at a focal point 53. It is intended that the focal point 53 of the piercing tip 50 can be pointed (e.g., conical or pyramidal) or blunt (e.g., flat, rounded, or bulbous).

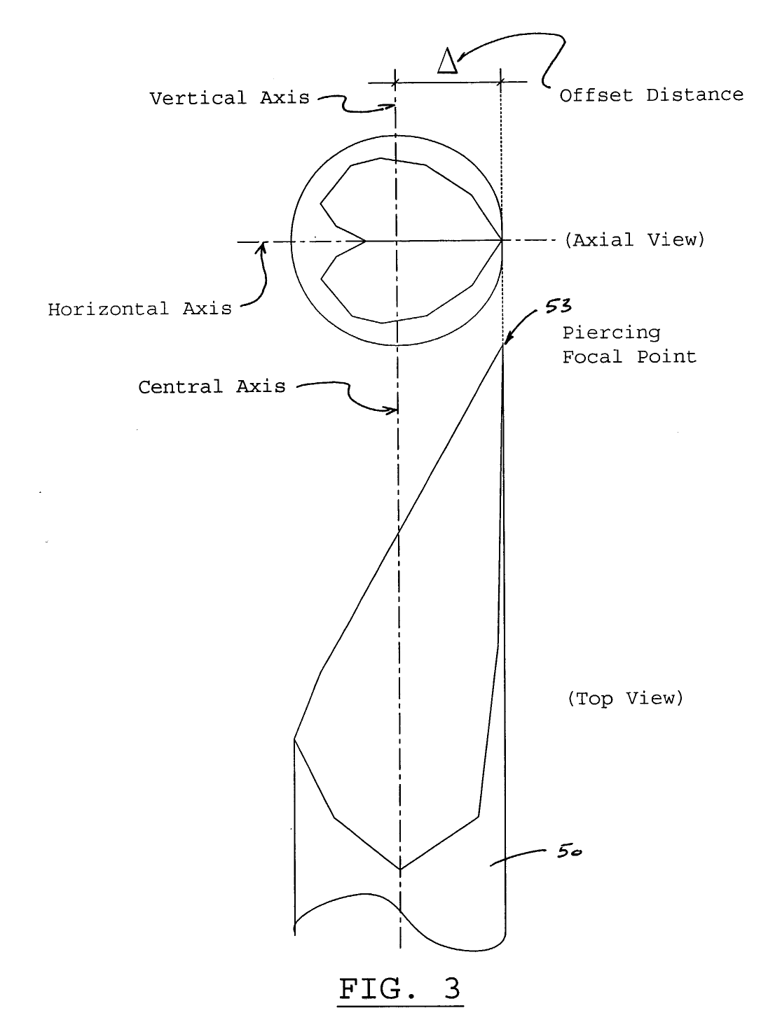

With reference to FIG. 3, the focal point 53 of the piercing tip 50 is shown in greater detail. The focal point 53 is offset along the horizontal axis of the central axis of the shaft at a distance A from the vertical axis of the central longitudinal axis of the shaft. The offset distance A is determined by the particular surgical application and the diameter of the obturator employed. While the offset distance A of the focus point 53 of the piercing tip 50 is shown in FIG. 3 to be substantially equal to the radius of the piercing tip such that the focus point is aligned with the perimeter of the obturator shaft, it is intended that a piercing tip in accordance with the present invention can be fabricated with an offset distance ranging from 1/64 of the radius of the piercing tip to the full radius of the piercing tip.

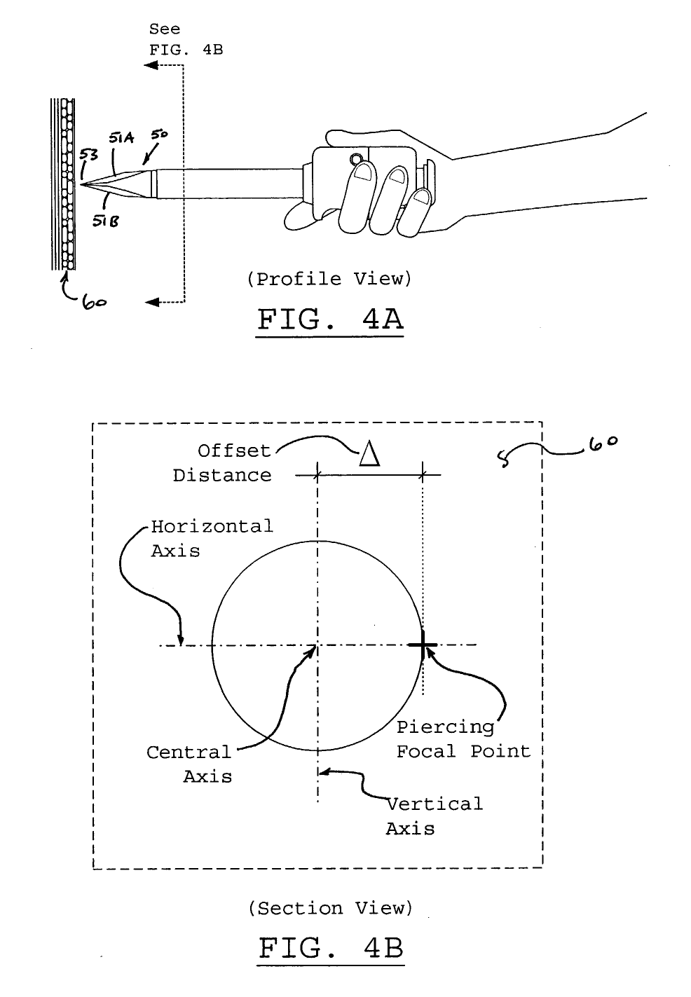

In operation, with reference to FIGS. 4A and 4B, the focal point 53 of the piercing tip 50 is aligned with an incision made by a surgeon in the abdominal wall 60 of a patient. Due to the flattened upper and lower faces 51A and 51B and the horizontally offset focal point 53 of the piercing tip, penetration trough the abdominal wall 60 is achieved by a side-to-side motion of the surgeon's hand and with minimal force applied perpendicular to the abdominal wall. This results in a cleaner and more controlled incision and a smaller risk to collateral tissue and organs than with conventional trocars which require a full rocking motion and a greater perpendicular force.

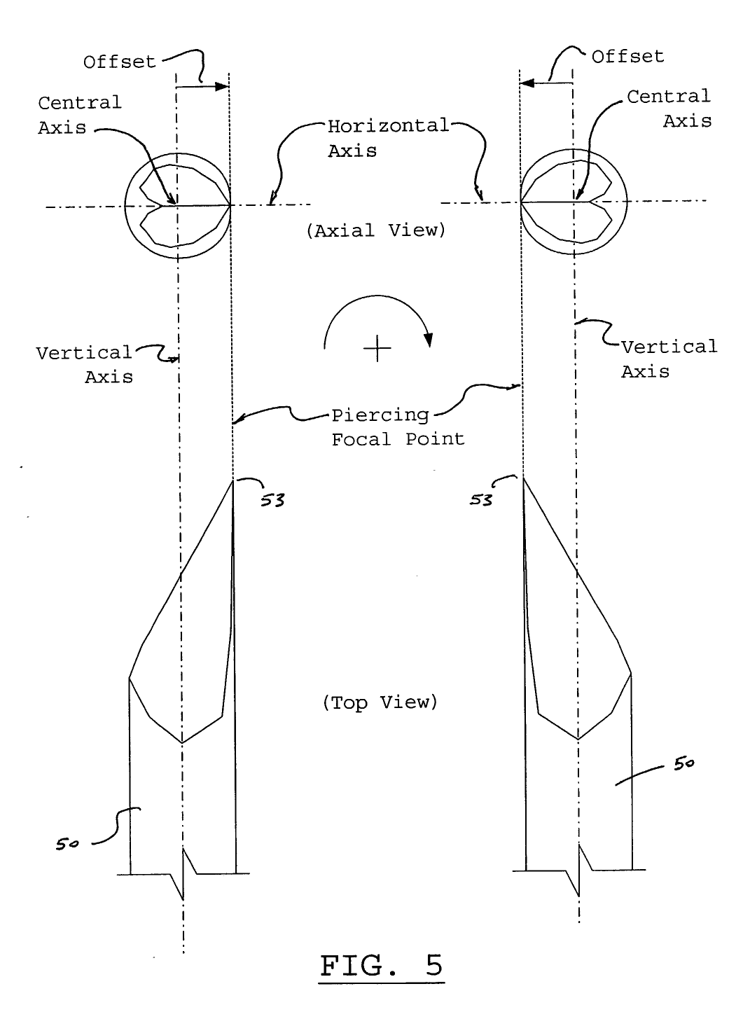

In another embodiment of the present invention, with reference to FIG. 5, the offset piercing tip 50 of the obturator can be rotated by the surgeon between: (1) a “right” position where the focal 53 point of the tip is located along the horizontal axis of the central axis and to the right of the vertical axis of the central axis of the obturator shaft, and (2) a “left” position where the focal point 53 of the tip is located along the horizontal axis of the central axis and to the left of the vertical axis of the central axis. This allows the surgeon to make the insertion from the left side or the right side of the patient.

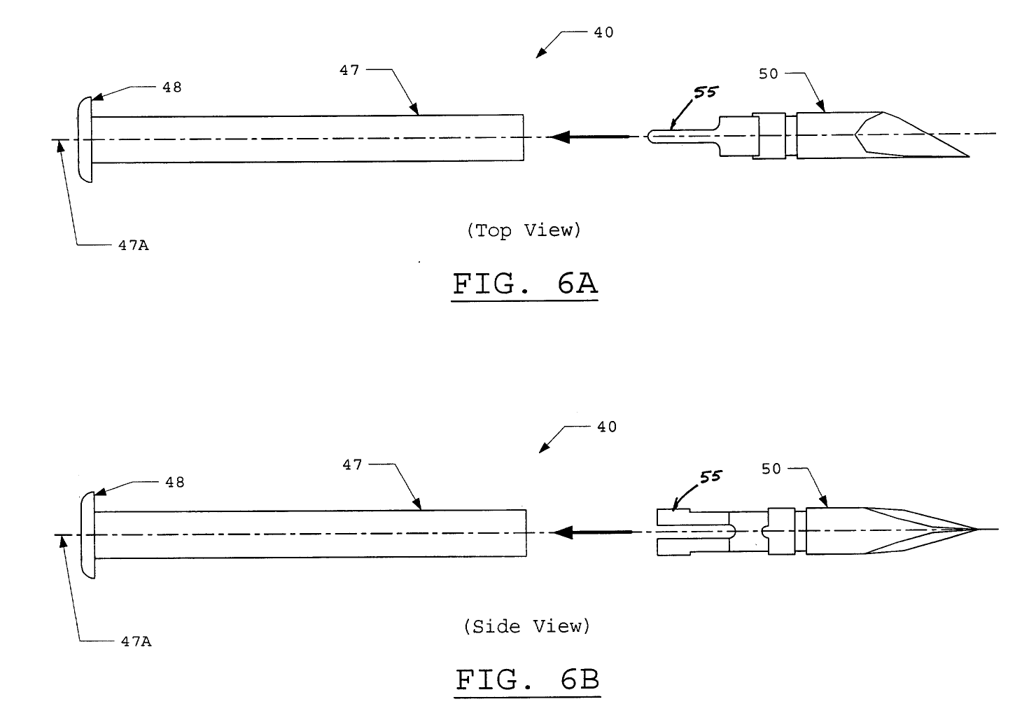

With reference to FIGS. 6A and 6B, in still another embodiment of the present invention, the piercing tip 50 of the obturator assembly 40 is replaceable. This replaceability allows the surgeon to employ different tips using the same trocar to accommodate a variety of procedures and conditions. For instance, the surgeon may interchange piercing tips between pointed, blunt, or bulbous tips. The techniques for making replaceable tips for trocars is well-known in the art, for example as shown in U.S. Pat. No. 5,697,947 to Wolf, which is incorporated herein by reference. But, generally, an obturator assembly 40 employing a replaceable piercing tip 50 includes a shaft 47 having an axial bore therethrough for receiving a set of clips 55 of the replaceable piercing tip. As the clips 55 of the piercing tip 50 are inserted into the obturator shaft 47, the clips engage the axial bore and lock the piercing tip into place for insertion into the abdominal wall of a patient.

In yet another embodiment of the present invention, the piercing tip is formed on the distal end of the obturator and is integral with the obturator such that the piercing tip cannot be separated and is not replaceable (See FIGS. 1A and 1B).

It should be understood that the examples and embodiments described herein are for illustrative purposes only and that various modifications or changes in light thereof will be suggested to persons skilled in the art and are to be included within the spirit and purview of this application and the scope of the appended claims. While the trocar of the present invention is described for use in penetrating the abdominal wall of a patient, it is intended that the invention can be employed for any medical procedure employing a trocar with a piercing tip.

What is claimed is:

1. A piercing tip for connection with an obturator shaft, said obturator shaft having a central longitudinal axis, said piercing tip comprising a substantially planar upper face, a substantially planar lower face, and an angled segment arranged between the upper face and the lower face; said upper face, lower face, and angled segment tapering away from the obturator shaft and converging to a focal point which is horizontally offset from the central longitudinal axis of the obturator shaft.

2. The piercing tip of claim 1, wherein the angled segment is beveled.

3. The piercing tip of claim 2, wherein the angled segment is blunt.

4. The piercing tip of claim 1, wherein the focal point is pointed.

5. The piercing tip of claim 1, wherein the focal point is blunt.

6. A trocar, comprising:
(a) a body assembly;
(b) a cannula assembly attached to the body assembly; said cannula assembly defining an axial bore therethrough; and
(c) an obturator assembly for sliding engagement in the axial bore, said obturator assembly having a central longitudinal axis and comprising a shaft having a distal end for insertion into a patient and a piercing tip arranged on the distal end of the shaft, said piercing tip comprising: (i) a substantially planar upper face, (ii) a substantially planar lower face, and (iii) an angled segment arranged between the upper face and the lower face, said upper face, lower face, and angled segment tapering away from the obturator shaft and converging to a focal point which is horizontally offset from the central longitudinal axis of the obturator shaft.

7. The trocar of claim 6, wherein the angled segment is beveled and non-cutting.

8. The trocar of claim 7, wherein the focal point is pointed.

9. The trocar of claim 7, wherein the focal point is blunt.

10. The trocar of claim 6, wherein the piercing tip of the obturator assembly is replaceable.

11. The trocar of claim 6, wherein the piercing tip is moveable between a right position whereby the focal point of the piercing tip is horizontally offset to the right of the central longitudinal axis and a left position whereby the focal point of the piercing tip is horizontally offset to the left of the central longitudinal axis.

12. A trocar, comprising:
(a) a body assembly;
(b) a cannula assembly attached to the body assembly; said cannula assembly defining an axial bore therethrough; and
(c) an obturator assembly for sliding engagement in the axial bore, said obturator assembly having a central longitudinal axis and comprising a shaft having a distal end for insertion into a patient and a piercing tip located on the distal end of the shaft, said piercing tip comprising: (i) a substantially planar upper face, (ii) a substantially planar lower face, and (iii) an angled segment arranged between the upper face and the lower face, said upper face, lower face, and angled segment tapering away from the obturator shaft and converging to a focal point which is horizontally offset from the central longitudinal axis of the obturator shaft, said angled segment being beveled with a non-cutting peak, said piercing tip being moveable between a right position whereby the focal point of the piercing tip is horizontally offset to the right of the central longitudinal axis and a left position whereby the focal point of the piercing tip is horizontally offset to the left of the central longitudinal axis.
